(19) United States
(12) Patent Application Publication
(10) Pub. No.: US 2026/0002901 A1
(43) Pub. Date: Jan. 1, 2026

(54) POLAR LIQUID SENSING APPARATUS

(71) Applicant: KOREA ELECTRONICS TECHNOLOGY INSTITUTE, Seongnam-si (KR)

(72) Inventor: Soon Hyung KWON, Seongnam-si (KR)

(73) Assignee: KOREA ELECTRONICS TECHNOLOGY INSTITUTE, Seongnam-si (KR)

(21) Appl. No.: 19/230,403

(22) Filed: Jun. 6, 2025

(30) Foreign Application Priority Data
Jun. 28, 2024 (KR) 10-2024-0085575

(51) Int. Cl. G01N 27/22; G01N 33/28

(52) U.S. Cl. CPC G01N 27/223; G01N 27/226; G01N 33/2847

(57) ABSTRACT

Disclosed is a polar liquid sensing apparatus including a sensor configured to output a voltage signal proportional to the content of a polar liquid in a liquid and a controller configured to determine that a non-polar liquid includes the polar liquid if the magnitude of the voltage signal exceeds a reference value, wherein the sensor may be a droplet type sensor or a dipping type sensor.

### CROSS REFERENCE TO RELATED APPLICATION

The present application claims priority to Korean Patent Application No. 10-2024-0085575, filed Jun. 28, 2024, the entire contents of which is incorporated herein for all purposes by this reference.

### TECHNICAL FIELD

The present disclosure relates to a polar liquid sensing apparatus.

### BACKGROUND

Liquids may be categorized into polar and non-polar liquids. A liquid may be classified as a polar liquid if polarity exists in the molecules constituting the liquid, and a liquid may be classified as a non-polar liquid if the molecules constituting the liquid are non-polar. Typically, water is classified as a polar liquid, and oil is classified as a non-polar liquid.

When a vehicle is filled with gasoline or diesel at a gas station, oil may be mixed with water. This may be caused by temperature differences as an oil storage tank is located underground in the gas station. If the oil storage tank is damaged, the oil may be mixed with water through the wall of the oil storage tank. If gasoline or diesel mixed with water enters the engine, the engine may fail. In order to prevent accidents that may occur as the result of oil being mixed with water, it is necessary to determine whether oil is mixed with water.

### SUMMARY

It is an aspect of the present disclosure to provide a polar liquid sensing apparatus capable of determining whether oil is mixed with water using a voltage signal generated by a droplet mixture of the oil and the water falling on a sensor.

According to an aspect of the present disclosure, a polar liquid sensing apparatus includes a sensor configured to output a voltage signal proportional to the content of a polar liquid in a liquid and a controller configured to determine that a non-polar liquid includes the polar liquid if the magnitude of the voltage signal exceeds a reference value.

According to an embodiment, the polar liquid may include water, and the non-polar liquid may include one of gasoline, diesel, kerosene, aviation fuel, marine fuel, crude oil, or edible oil.

According to an embodiment, the sensor may include a container configured to store a liquid, a nozzle configured to discharge the liquid stored in the container in the form of a droplet, a substrate located at an angle with respect to a direction in which the droplet falls from the nozzle, a first electrode formed on the substrate, an energy conversion layer formed on the substrate while covering the first electrode, and a second electrode formed on the energy conversion layer so as to be spaced apart from the first electrode, wherein the first electrode and the second electrode may generate a voltage signal proportional to the amount of the polar liquid included in the droplet while the droplet flows over the substrate.

According to an embodiment, the second electrode may be formed on the substrate so as to be spaced apart from the first electrode, and the energy conversion layer may be formed on the substrate while covering the first electrode and the second electrode.

According to an embodiment, the sensor may include an energy conversion layer formed in the shape of a container configured to store a liquid, a first electrode formed on an outer surface of the energy conversion layer, a second electrode located inwardly of the container, and a driving unit configured to perform an operation of immersing one end of the second electrode in the liquid to a predetermined depth and an operation of withdrawing the second electrode from the liquid, wherein the first electrode and the second electrode may generate a voltage signal proportional to the amount of the polar liquid included in the liquid while the one end of the second electrode is immersed in or withdrawn from the liquid.

According to an embodiment, a unit electrode constituted by the first electrode and the second electrode may be provided in plural so as to be disposed side by side in a direction in which the droplet flows, and the controller may determine that the liquid includes the polar liquid if the average of magnitudes of voltage signals output by the plurality of unit electrodes exceeds the reference value.

According to an embodiment, a unit structure including the energy conversion layer, the first electrode, the second electrode, and the driving unit may be provided in plural so as to be disposed side by side in a lateral direction of the container, and the controller may determine that the non-polar liquid includes the polar liquid if the average of magnitudes of voltage signals output by the plurality of unit structures exceeds the reference value.

According to an embodiment, the controller may perform a plurality of measurements using the sensor and may determine that the liquid includes the polar liquid if the average of magnitudes of a plurality of voltage signals exceeds the reference value.

According to an embodiment, the controller may determine the content of the polar liquid included in the droplet by determining within which of a plurality of voltage ranges matched to pre-stored contents of the polar liquid the magnitude of the voltage signal generated by the sensor falls.

According to an embodiment, the polar liquid sensing apparatus may further include a display unit configured to display that the non-polar liquid includes the polar liquid to a user based on a control signal that the controller outputs upon determining that the non-polar liquid includes the polar liquid.

The features and advantages of the present disclosure will be more clearly understood from the following detailed description taken in conjunction with the accompanying drawings.

It should be understood that the terms used in the specification and appended claims should not be construed as being limited to general and dictionary meanings, but should be construed based on meanings and concepts according to the spirit of the present disclosure on the basis of the principle that the inventor is permitted to define appropriate terms for the best explanation.

### BRIEF DESCRIPTION OF THE DRAWINGS

The above and other objects, features and other advantages of the present disclosure will be more clearly understood from the following detailed description taken in conjunction with the accompanying drawings, in which:

FIG. 1 is a view showing a polar liquid sensing apparatus according to an embodiment;

FIG. 2 is a view showing a droplet type sensor according to an embodiment;

FIG. 3 is a view showing the positions of a first electrode, a second electrode, and an energy conversion layer according to an embodiment;

FIG. 4 is a view showing a structure in which both the first electrode and the second electrode according to the embodiment are covered by the energy conversion layer;

FIG. 5 is a view showing a droplet type sensor in which a plurality of unit electrodes according to an embodiment is arranged;

FIG. 6 is a view showing a dipping type sensor according to an embodiment;

FIG. 7 is a view showing the operation of the dipping type sensor according to the embodiment; and

FIG. 8 is a view showing the operation of a dipping type sensor in which a plurality of unit structures according to an embodiment is arranged.

### DETAILED DESCRIPTION

Hereinafter, the present disclosure will be described in detail (with reference to the accompanying drawings). However, this is by way of example only, and the present disclosure is not limited to a specific embodiment described as an example.

Hereinafter, an embodiment of the present disclosure will be described in detail with reference to the accompanying drawings.

FIG. 1 is a view showing a polar liquid sensing apparatus 10 according to an embodiment.

According to the embodiment, the polar liquid sensing apparatus 10 may include a sensor 20 configured to output a voltage signal proportional to the content of a polar liquid 1 in a liquid and a controller 30 configured to determine that the polar liquid 1 is included in a non-polar liquid 2 if the magnitude of the voltage signal exceeds a reference value.

The polar liquid 1 may include water, and the non-polar liquid 2 may include one of gasoline, diesel, kerosene, aviation fuel, marine fuel, crude oil, or edible oil. The non-polar liquid 2 may be mixed with the polar liquid 1 for a variety of reasons. When the non-polar liquid 2 utilized for a given purpose is mixed with the polar liquid 1, the non-polar liquid 2 may not exhibit expected performance. The polar liquid sensing apparatus according to the embodiment has a simple structure, is easy to manufacture, and requires a short time for inspection, whereby it is possible to easily inspect whether the non-polar liquid 2 is mixed with the polar liquid 1. For example, if oil from a gas station is put in the sensor 20, it is possible to rapidly check whether the oil is mixed with water.

The sensor 20 may include a first electrode 110, a second electrode 120, and an energy conversion layer 130 located between the first electrode 110 and the second electrode 120. When a mixed liquid 3, which is a mixture of the polar liquid 1 and the non-polar liquid 2, comes into contact with the energy conversion layer 130, the energy conversion layer 130 may be electrically charged depending on the content of the polar liquid 1. Accordingly, a voltage signal may be generated between the first electrode 110 and the second electrode 120. That is, a voltage signal may be generated by triboelectrification.

The controller 30 may receive the voltage signal generated by the first electrode 110 and the second electrode 120. The controller 30 may include an A/D converter configured to measure the magnitude of the voltage signal, a processor, a memory, and an input/output interface configured to receive input from a user and to output information.

The controller 30 may compare the magnitude of the voltage signal to a reference value and determine that the polar liquid 1 is included in the non-polar liquid 2 if the magnitude of the voltage signal is higher than the reference value. The controller 30 may determine that the polar liquid 1 is not included in the non-polar liquid 2 if the magnitude of the voltage signal is equal to or less than the reference value.

Conversely, if the non-polar liquid 2 is included in the polar liquid 1, the sensor 20 may output a voltage signal smaller than the voltage signal that occurs when only the polar liquid 1 is present. Accordingly, the controller 30 may compare the voltage signal to the reference value to determine whether the polar liquid 1 is mixed with the non-polar liquid 2. Although determination as to whether the non-polar liquid 2 is mixed with the polar liquid 1 is described herein, it may be understood that it is also possible to determine whether the polar liquid 1 is mixed with the non-polar liquid 2.

According to an embodiment, the polar liquid sensing apparatus 10 may further include a display unit 40 configured to display that the non-polar liquid 2 includes the polar liquid 1 to a user based on a control signal that the controller 30 outputs upon determining that the non-polar liquid 2 includes the polar liquid 1.

The display unit 40 may include a display or a speaker. The controller 30 may display whether the polar liquid 1 is included to the user through the display unit 40. The controller 30 may inform a predetermined target of whether the polar liquid 1 is included through a wired or wireless network.

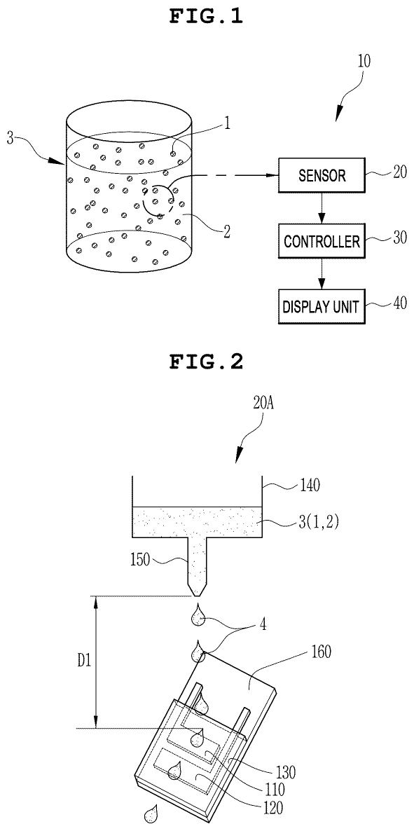

FIG. 2 is a view showing a droplet type sensor 20A according to an embodiment.

The droplet type sensor 20A may include a container 140 configured to store a liquid, a nozzle 150 configured to discharge the liquid stored in the container 140 in the form of a droplet 4, a substrate 160 located at an angle with respect to the direction in which the droplet falls from the nozzle 150, a first electrode 110 formed on the substrate 160, an energy conversion layer 130 formed on the substrate 160 while covering the first electrode 110, and a second electrode 120 formed on the energy conversion layer 130 so as to be spaced apart from the first electrode 110, wherein the first electrode 110 and the second electrode 120 may generate a voltage signal proportional to the amount of the polar liquid 1 included in the droplet 4 while the droplet 4 flows over the substrate 160.

The container 140 may at least temporarily store the liquid. The liquid stored in the container 140 is a liquid that the user wishes to test in order to determine whether the liquid is mixed with the polar liquid 1. The user may introduce the non-polar liquid 2 that may include the polar liquid 1 into the container 140. The user may put the polar liquid 1 that may include the non-polar liquid 2 into the container 140. The user may put the mixed liquid 3, which is the mixture of the polar liquid 1 and the non-polar liquid 2, into the container 140.

The nozzle 150 may be connected to a lower part of the container 140. The nozzle 150 may cause the liquid stored in the container 140 to fall in the form of a droplet 4. The nozzle 150 may be formed in the shape of a narrow tube connected to the container 140. The length and diameter of the nozzle 150 may be determined by the properties of the polar liquid 1 or non-polar liquid 2 to be measured. The droplet 4 falling from the nozzle 150 may be directed to the substrate 160 on which the first electrode 110 and the second electrode 120 are formed.

The substrate 160 may be located at an angle such that the droplet 4 falling from the nozzle 150 flows along one surface thereof. The first electrode 110, the second electrode 120, and the energy conversion layer 130 may be formed on the substrate 160. The distance between the substrate 160 and the container 140 or the nozzle 150 may be relatively fixed such that the distance D1 between the end of the nozzle 150 and the point where the droplet 4 falls is fixed. Thus, the distance D1 until the droplet 4 falling from the nozzle 150 comes into contact with the substrate 160 may remain constant.

The substrate 160 may be made of silicone, glass, a polymer material, or ceramic. The polymer substrate 160 may be a plastic substrate 160 or film including at least one of polyethylene terephthalate (PET), polyarylate (PAR), polymethylmethacrylate (PMMA), or polyethylene naphthalate (PEN), polyethersulfone (PES), polyimide (PI), polycarbonate (PC), and fiber reinforced plastic (FRP).

The ceramic substrate 160 may be made of a ceramic material including at least one of alumina (Al₂O₃), beryllia (BeO), aluminum nitride (AlN), silicon carbide, mullite, and silicon. In addition, the substrate 160 may be made of a fabric material such as nylon, cotton, or polyester.

Each of the first electrode 110 and the second electrode 120 may be made of an electrically conductive material. The first electrode 110 and the second electrode 120 may constitute a unit electrode 170. The unit electrode causes a polarization phenomenon through a change in the contact state, including any one of the contact angle, contact surface, and contact area with the electrodes, due to a predetermined flow of a liquid droplet on the first electrode 110 and the second electrode 120 patterned on the substrate 160 so as to be spaced apart from each other and generates electrical energy thereby. The first electrode 110 or the second electrode 120 may be not only a conductive metal electrode but also a conductive fabric electrode formed by coating a fabric with copper, nickel, silver, or the like.

The first electrode 110 or the second electrode 120 may be an inorganic electrode including at least one of ITO, IGO, chromium, aluminum, indium zinc oxide (IZO), indium gallium zinc oxide (IGZO), ZnO, ZnO₂, and TiO₂, a metal electrode including at least one of platinum, gold, silver, aluminum, iron, and copper, or an organic electrode including at least one of polyethylenedioxythiophene (PEDOT), carbon nanotube (CNT), graphene, polyacetylene, polythiophene (PT), polypyrrole, polyparaphenylene (PPV), polyaniline, poly sulfur nitride, stainless steel, a ferrous alloys containing at least 10% of chromium, SUS 304, SUS 316, SUS 316L, a Co—Cr alloy, a Ti alloy, nitinol (Ni—Ti), and poly(p-phenylene vinylene).

The energy conversion layer 130 is formed by stacking an inorganic layer and/or an organic layer. Preferably, the energy conversion layer 130 may be formed by patterning, deposition, or spin coating.

The energy conversion layer 130 may include an organic layer including at least one of polymethylmethacrylate (PMMA), polyethylene (PE), polystyrene (PS), polyvinylpyrrolidone (PVP), poly(4-vinylpenol) (PVP), polyethersulfone (PES), poly(4-methoxyphenylacrylate) (PMPA), poly(phenylacrylate) (PPA), poly(2,2,2-trifluoroethyl methacrylate) (PTFMA), cyanoethyl pullulan (CYEPL), polyvinyl chloride (PVC), a poly(parabanic acid) resin (PPA), poly(t-butylstyrene) (PTBS), polythienylenevinylene (PTV), polyvinylacetate (PVA), poly(vinyl alcohol) (PVA), poly(α-methylstyrene) (PAMS), poly(vinylalcohol)-co-poly (vinyl acetate)-co-poly(itaconic acid) (PVAIA), polyolefin, polyacrylate, parylene-C, polyimide, octadecyltrichlorosilane (OTS), poly(triarylamine) (PTTA), poly-3-hexylthiophene (P3HT), cross-linked poly-4-vinylphenol (cross-linked PVP), poly(perfluoroalkenylvinyl ether), nylon-6, n-octadecylphosphonic acid (ODPA), polytetrafluoroethylene (PTFE), silicone, polyurethane, latex, cellulose acetate, poly(hydroxy ethyl methacrylate) (PHEMA), polylactide (PLA), polyglycolide (PGA), and polyglycolide-co-lactide (PGLA), and an inorganic layer including at least one of silicon oxide (SiO₂), titanium oxide (TiO₂), aluminum oxide (Al₂O₃), tantalum (Ta₂O₅), tantalum pentoxide, zinc oxide (ZnO), tantalum pentoxide, Ta₂O₅), yttrium oxide (Y₂O₃), cerium oxide (CeO₂), titanium dioxide (TiO₂), barium titanate (BaTiO₃), barium zirconate titanate (BZT), zirconium dioxide (ZrO₂), lanthanum oxide (La₂O₃), hafnium silicate (Hafnon, HfSiO₄), lanthanum aluminate (LaAlO₃), silicon nitride (Si₃N₄), and strontium titanate (SrTiO₃), barium strontium titanate (BST), lead zirconate titanate (PZT), calcium copper titanate (CCTO), hafnium oxide (HfO₂), apatite (A₁₀(MO₄)₆(X)₂), hydroxyapatite (Ca₁₀(PO₄)₆(OH)₂), tricalcium phosphate (Ca₃(PO₄₂)), Na₂OCaO—SiO₂, and bioglass (CaO—SiO₂—P₂O₅), as perovskite materials. Furthermore, polytetrafluoroethylene, ethylene-tetrafluoroethylene, fluorinated ethylene propylene (FEP), or a perfluoroalkoxy copolymer may also be used.

The organic layer may be made of a material with a dielectric constant (K) of 4 or less, and the inorganic layer may be made of a material with a dielectric constant (K) of 5 or more. The inorganic layer and the organic layer may be stacked on the first electrode 110 or the second electrode 120 in any order, but are preferably stacked adjacent to each other. The energy conversion layer 130 may be formed by repeatedly stacking the inorganic layer and the organic layer.

The unit electrode including the first electrode 110 and the second electrode 120 and the energy conversion layer 130 generate energy in response to a change in the contact state of the droplet 4. As the droplet 4 flows over the first electrode 110 or the second electrode 120, the resulting change in contact state causes a change in capacitance, and a voltage signal may be generated from the movement of electrons that occurs to compensate for the resulting potential difference.

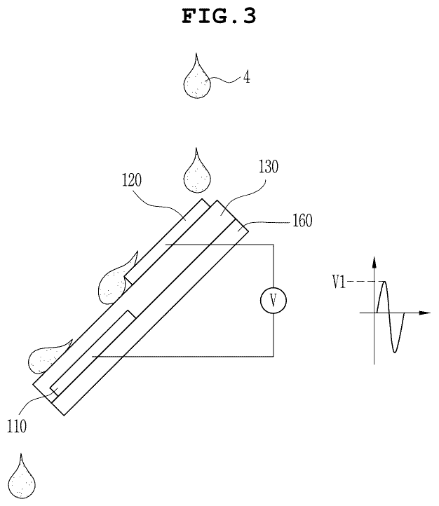

FIG. 3 is a view showing the positions of a first electrode 110, a second electrode 120, and an energy conversion layer 130 according to an embodiment.

The first electrode 110 may be formed on the substrate 160. The energy conversion layer 130 may be formed on the substrate 160 so as to cover the first electrode 110. The second electrode 120 may be formed on the energy conversion layer 130. Since the second electrode 120 is formed on the energy conversion layer 130, the droplet 4 may directly contact the second electrode 120.

A voltage signal may be generated as the droplet 4 contacts the second electrode 120, and a voltage signal in the opposite direction may be generated as the droplet 4 leaves the second electrode 120 and passes through the first electrode 110. Such a voltage signal V1 may be generated between the first electrode 110 and the second electrode 120 and input to the controller 30.

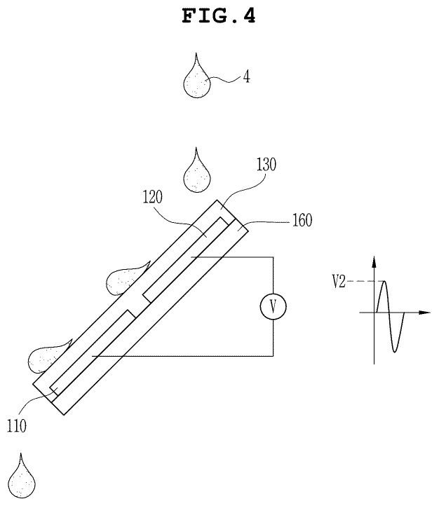

FIG. 4 is a view showing a structure in which both the first electrode 110 and the second electrode 120 according to the embodiment are covered by the energy conversion layer 130.

The first electrode 110 may be formed on the substrate 160, and the second electrode 120 may be formed on the substrate 160 so as to be spaced apart from the first electrode 110. The energy conversion layer 130 may be formed on the substrate 160 while covering the first electrode 110 and the second electrode 120. After the first electrode 110 and the second electrode 120 are formed on the substrate 160 so as to be spaced apart from each other by a predetermined distance, the energy conversion layer 130 may be formed so as to cover both the first electrode 110 and the second electrode 120.

A voltage signal may be generated as the droplet 4 falls and passes through the second electrode 120, and a voltage signal in the opposite direction may be generated as the droplet 4 leaves the second electrode 120 and passes through the first electrode 110. Such a voltage signal V2 may be generated between the first electrode 110 and the second electrode 120 and input to the controller 30.

Both the structure shown in FIG. 3 and the structure shown in FIG. 4 may be used. Since the droplet 4 can directly contact the second electrode 120 in the structure shown in FIG. 3, the polar liquid 1 in the droplet 4 may directly contact the second electrode 120, whereby the magnitude of the voltage signal V1 may be greater than the magnitude of the voltage signal V2 generated in the structure of FIG. 4.

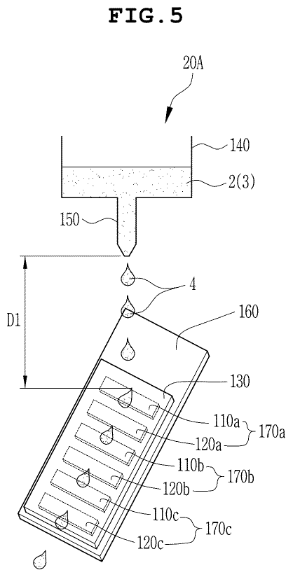

FIG. 5 is a view showing a droplet type sensor 20A in which a plurality of unit electrodes 170 according to an embodiment is arranged.

The unit electrode 170, which is constituted by the first electrode 110 and the second electrode 120, may be provided in plural so as to be disposed side by side in the direction in which the droplet 4 flows. The plurality of unit electrodes 170a, 170b, and 170c may be formed on the substrate 160. The plurality of unit electrodes 170a, 170b, and 170c may be formed in the structure shown in FIG. 3 or 4. The plurality of unit electrodes 170a, 170b, and 170c may be located side by side along the slope of the substrate 160 in the direction in which the droplet 4 flows. That is, one droplet 4 may pass through the plurality of unit electrodes 170a, 170b, and 170c.

When the droplet 4 passes through the plurality of unit electrodes 170a, 170b, and 170c, a voltage signal may be output by each of the unit electrodes 170a, 170b, and 170c. For example, the first unit electrode 170a may include a first electrode 110a and a second electrode 120a, and may output a voltage signal Va. The second unit electrode 170b includes a first electrode 110b and a second electrode 120b, and may output a voltage signal Vb. The third unit electrode 170c includes a first electrode 110c and a second electrode 120c, and may output a voltage signal Vc.

The controller 30 may determine that the liquid includes the polar liquid 1 if the average of the magnitudes of the voltage signals output by the plurality of unit electrodes 170 exceeds a reference value. Since a measurement error may occur for various reasons, the controller 30 may reduce the error by averaging the voltage signals output by the plurality of unit electrodes 170.

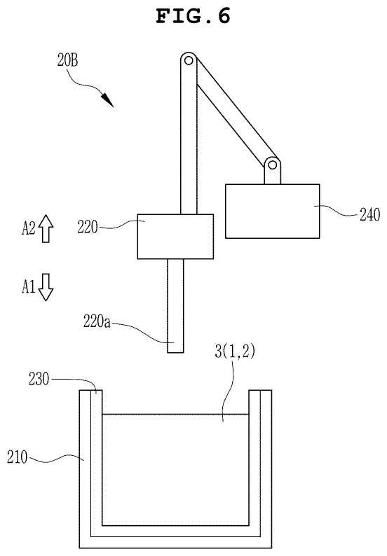

FIG. 6 is a view showing a dipping type sensor 20B according to an embodiment.

The dipping type sensor 20B according to the embodiment includes an energy conversion layer 230 formed in the shape of a container configured to store a liquid, a first electrode 210 formed on an outer surface of the energy conversion layer 230, a second electrode 220 located inwardly of the container, and a driving unit 240 configured to perform an operation of immersing one end of the second electrode 220 in the liquid to a predetermined depth and an operation of withdrawing the second electrode 220 from the liquid, wherein the first electrode 210 and the second electrode 220 may generate a voltage signal proportional to the amount of the polar liquid 1 included in the liquid during the processes in which the end of the second electrode 220 is immersed in or withdrawn from the liquid.

The energy conversion layer 230 may be formed entirely in the shape of a container capable of storing the liquid. The liquid may be at least temporarily stored in the energy conversion layer 230, and the first electrode 210 may be formed outside the energy conversion layer 230. The first electrode 210 may be formed so as to cover the outer surface of the energy conversion layer 230. The second electrode 220 may be spaced apart from the first electrode 210, and the second electrode 220 may be immersed in the liquid stored in the container-shaped energy conversion layer 230.

When one end of the second electrode 220 is immersed in the liquid, the electrical capacity between the first electrode 210 and the second electrode 220 may be changed according to a change in at least one of the contact surface, the contact angle, and the contact area where the liquid and the energy conversion layer 230 are in contact, whereby a voltage signal may be generated. When one end of the second electrode 220 is immersed in the liquid, the contact area between the energy conversion layer 230 and the liquid changes and the contact angle therebetween changes, which may generate a voltage signal between the first electrode 210 and the second electrode 220.

The material of each of the first electrode 210, the second electrode 220, and the energy conversion layer 230 has been described above, and therefore a description thereof will be omitted.

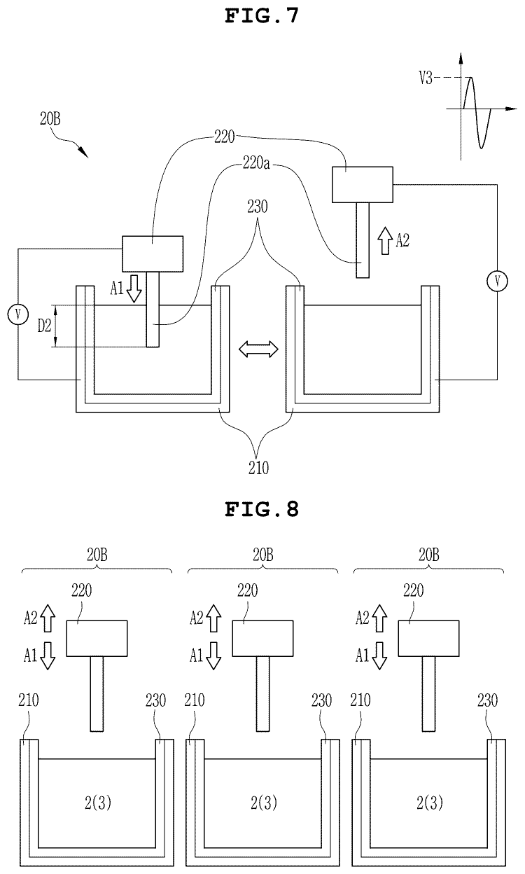

The depth D1 to which the second electrode 220 is immersed in the liquid is a predetermined value. The second electrode 220 may be moved by the driving unit 240. The driving unit 240 may move the second electrode 220 in the direction A1 in which the second electrode 220 is immersed in the liquid, or may move the second electrode 220 in the direction A2 in which the second electrode 220 is withdrawn from the liquid. The driving unit 240 may include a motor, an arm, a gear, and a shaft. FIG. 7 is a view showing the operation of the dipping type sensor 20B according to the embodiment.

The driving unit 240 of the dipping type sensor 20B may repeatedly immerse the second sensor 20 in the liquid to the predetermined depth and withdraw the second sensor 20 from the liquid. When one end of the second sensor 20 is immersed in the liquid to the predetermined depth D2, a voltage signal may be generated between the first electrode 210 and the second electrode 220. When one end of the second sensor 20 is withdrawn from the liquid, a voltage signal in the opposite direction may be generated between the first electrode 210 and the second electrode 220.

The controller 30 may receive the voltage signal generated by the first electrode 210 and the second electrode 220. The controller 30 may compare the magnitude of the voltage signal to a reference value, and determine that the polar liquid 1 is included if the magnitude of the voltage signal is higher than the reference value. The controller 30 may determine that the polar liquid 1 is not included if the magnitude of the voltage signal is equal to or less than the reference value.

FIG. 8 is a view showing the operation of a dipping type sensor 20B in which a plurality of unit structures according to an embodiment is arranged.

A plurality of unit structures, each of which includes an energy conversion layer 230, a first electrode 210, a second electrode 220, and a driving unit 240, may be disposed side by side in a lateral direction of the container 140. One driving unit 240 may actuate the plurality of second electrodes 220 of the plurality of unit structures. The plurality of first electrodes 210 may be spaced apart from each other between the unit structures. The plurality of second electrodes 220 may also be spaced apart from each other. In effect, one unit structure may be one dipping type sensor 20B, and the plurality of unit structures may be a plurality of dipping type sensors 20B.

The controller 30 may determine that the polar liquid 1 is included in the non-polar liquid 2 if the average of the magnitudes of the voltage signals output by the plurality of unit structures exceeds the reference value. Since a measurement error may occur for various reasons, the controller 30 may reduce the error by averaging the voltage signals output by the plurality of unit structures.

As described above, the polar liquid sensing apparatus 10 according to the embodiment may use two structures: the droplet type sensor 20A and the dipping-type sensor 20B. Regardless of the type of sensor 20, the controller 30 may perform a plurality of measurements and average the magnitudes of a plurality of voltage signals.

The controller 30 may perform a plurality of measurements using the sensor 20 and determine that the liquid includes the polar liquid 1 if the average of the magnitudes of a plurality of voltage signals exceeds the reference value.

For example, even if the plurality of unit electrodes 170 is not formed, the droplet type sensor 20A may output a voltage signal a plurality of times when the droplet 4 falls from the nozzle 150 a plurality of times. In addition, even if the plurality of unit structures (the dipping type sensors 20B) is not formed, the dipping type sensor 20B may output a voltage signal a plurality of times when the driving unit 240 performs the operation of dipping the second electrode 220 a plurality of times. The controller 30 may receive a plurality of voltage signals that is output in sequence, at least temporarily store the same, and average the plurality of voltage signals.

The controller 30 may determine the content of the polar liquid 1 included in the droplet 4 by determining within which of the plurality of voltage ranges matched to the pre-stored contents of the polar liquid 1 the magnitude of the voltage signal generated by the sensor 20 falls.

The controller 30 may store a plurality of voltage ranges. The plurality of voltage ranges may be matched with the content of the polar liquid 1 corresponding thereto. If the magnitude of the voltage signal received from the sensor 20 falls within a particular voltage range, the controller 30 may determine that the liquid includes the content of the polar liquid 1 that is matched to the specific voltage range.

The content of the polar liquid 1 may also be expressed as a range. For example, if the voltage ranges from x1 (V) to x2 (V), the content of the polar liquid 1 may be matched as ranging from y1 (%) to y2 (%). The controller 30 may display the content of the polar liquid 1 to the user through the display unit 40.

The controller 30 may compare the maximum magnitude of the voltage signal to the reference value. The controller 30 may receive the voltage signal output by the sensor 20 using an ADC, at least temporarily memorize the maximum magnitude, and compare the same to the reference value.

Since the magnitude of the voltage signal output by the sensor 20 is proportional to the content of the polar liquid 1, the reference value used to determine whether the non-polar liquid 2 includes the polar liquid 1 is different from the reference value used to determine whether the polar liquid 1 includes the non-polar liquid 2. Furthermore, in order to determine whether the polar liquid 1 includes the non-polar liquid 2, the controller 30 may determine that the polar liquid 1 includes the non-polar liquid 2 if the magnitude of the voltage signal is less than the reference value when the controller 30 compares the magnitude of the voltage signal with the reference value. The reference value may be determined differently depending on the application in which the polar liquid 1 and the non-polar liquid 2 are used. For example, about 2% water in oil at a gas station may not be a relatively large problem, but about 20% water in oil may be a problem. Thus, the reference value may be predetermined based on the nature of the liquid to be tested.

When the polar liquid sensing apparatus 10 according to the embodiment described above is used, a user may conveniently and rapidly determine if oil is mixed with water. Conversely, the user may also determine whether water is mixed with oil. The sensor 20 may be configured such that the first electrode 110 or 210, the second electrode 120 or 220, and the energy conversion layer 130 or 230 can be replaced so as to be used for one-time use. The disposable sensor 20 may be mass-produced at lower unit cost.

As is apparent from the above description, according to an embodiment of the present disclosure, it is possible to easily determine whether oil, such as gasoline or kerosene, is mixed with water.

According to the embodiment of the present disclosure, it is possible to measure the amount of the water in the oil such as gasoline or kerosene.

The present disclosure has been described in detail with reference to the specific embodiment. The above description is merely an example of applying the principles of the present disclosure, and other configurations may be included without departing from the scope of the present disclosure.

What is claimed is:

1. A polar liquid sensing apparatus comprising:
a sensor configured to output a voltage signal proportional to a content of a polar liquid in a liquid; and
a controller configured to determine that a non-polar liquid comprises the polar liquid if a magnitude of the voltage signal exceeds a reference value.

2. The polar liquid sensing apparatus according to claim 1, wherein
the polar liquid comprises water, and
the non-polar liquid comprises one of gasoline, diesel, kerosene, aviation fuel, marine fuel, crude oil, or edible oil.

3. The polar liquid sensing apparatus according to claim 1, wherein the sensor comprises:
a container configured to store a liquid;
a nozzle configured to discharge the liquid stored in the container in a form of a droplet;
a substrate located at an angle with respect to a direction in which the droplet falls from the nozzle;
a first electrode formed on the substrate;
an energy conversion layer formed on the substrate while covering the first electrode; and
a second electrode formed on the energy conversion layer so as to be spaced apart from the first electrode, and
the first electrode and the second electrode generate a voltage signal proportional to an amount of the polar liquid included in the droplet while the droplet flows over the substrate.

4. The polar liquid sensing apparatus according to claim 3, wherein
the second electrode is formed on the substrate so as to be spaced apart from the first electrode, and
the energy conversion layer is formed on the substrate while covering the first electrode and the second electrode.

5. The polar liquid sensing apparatus according to claim 1, wherein the sensor comprises:
an energy conversion layer formed in a shape of a container configured to store a liquid;
a first electrode formed on an outer surface of the energy conversion layer;
a second electrode located inwardly of the container; and
a driving unit configured to perform an operation of immersing one end of the second electrode in the liquid to a predetermined depth and an operation of withdrawing the second electrode from the liquid, and
the first electrode and the second electrode generate a voltage signal proportional to an amount of the polar liquid included in the liquid while the one end of the second electrode is immersed in or withdrawn from the liquid.

6. The polar liquid sensing apparatus according to claim 3, wherein
a unit electrode constituted by the first electrode and the second electrode is provided in plural so as to be disposed side by side in a direction in which the droplet flows, and
the controller determines that the liquid comprises the polar liquid if an average of magnitudes of voltage signals output by the plurality of unit electrodes exceeds the reference value.

7. The polar liquid sensing apparatus according to claim 5, wherein
a unit structure comprising the energy conversion layer, the first electrode, the second electrode, and the driving unit is provided in plural so as to be disposed side by side in a lateral direction of the container, and
the controller determines that the non-polar liquid comprises the polar liquid if an average of magnitudes of voltage signals output by the plurality of unit structures exceeds the reference value.

8. The polar liquid sensing apparatus according to claim 1, wherein the controller performs a plurality of measurements using the sensor and determines that the liquid comprises the polar liquid if an average of magnitudes of a plurality of voltage signals exceeds the reference value.

9. The polar liquid sensing apparatus according to claim 1, wherein the controller determines the content of the polar liquid included in the droplet by determining within which of a plurality of voltage ranges matched to pre-stored contents of the polar liquid the magnitude of the voltage signal generated by the sensor falls.

10. The polar liquid sensing apparatus according to claim 1, further comprising a display unit configured to display that the non-polar liquid comprises the polar liquid to a user based on a control signal that the controller outputs upon determining that the non-polar liquid comprises the polar liquid.
